(19) United States
(12) Patent Application Publication
(10) Pub. No.: US 2026/0166068 A1
(43) Pub. Date: Jun. 18, 2026

(54) COMPOSITION FOR PROMOTING DEVELOPMENT OF BRAIN FUNCTIONS IN CHILDREN

(71) Applicants: Meiji Co., Ltd., Tokyo (JP); Meiji Holdings Co., Ltd., Tokyo (JP)

(72) Inventors: Yoshitaka NAKAMURA, Tokyo (JP); Keigo SATO, Tokyo (JP); Shinji JINNO, Tokyo (JP); Masashi MORIFUJI, Tokyo (JP); Yoshie SAITO, Tokyo (JP)

(73) Assignees: Meiji Co., Ltd., Tokyo (JP); Meiji Holdings Co., Ltd., Tokyo (JP)

(21) Appl. No.: 19/116,552

(22) PCT Filed: Sep. 29, 2023
(86) PCT No.: PCT/JP2023/035563

§ 371 Date: Mar. 28, 2025

(30) Foreign Application Priority Data
Sep. 30, 2022 (JP) 2022-157391
Jul. 3, 2023 (JP) 2023-109619

(51) Int. Cl. A61K 31/706; A61P 25/00; A61P 25/28

(52) U.S. Cl. CPC A61K 31/706; A61P 25/00; A61P 25/28

(57) ABSTRACT

The effects of NMN on infants are clarified. The object is to provide a composition that can promote development of brain functions in infants or children. There is provided a composition for promoting development of a brain function in an infant or child, the composition comprising a nicotinamide adenine dinucleotide (NAD)-related substance or a salt thereof acceptable for use in foods or pharmaceuticals. The development of a brain function is preferably development in any area selected from the group consisting those of communication, gross motor, fine motor, problem solving, and personal-social.

### TECHNICAL FIELD

The present invention relates to a composition for promoting development of a brain function in an infant or child. The present invention is useful in the fields of development, health, and nutrition of infants and young children, as well as in the field of manufacturing foods or drinks for them, etc.

### BACKGROUND ART

Nicotinamide mononucleotide (NMN) is a precursor of nicotinamide adenine dinucleotide (NAD+), which is necessary for humans to maintain vital functions. Since the amount of NAD in the body declines with age, it is thought that taking NMN, a precursor of NAD, may be an effective way to cope with such decline.

For example, Non-patent documents 1 and 2 show that restoration of NAD+ levels in the brain may promote neurovascular coupling, on the basis of the results of experiments in which NMN was administered to aged mice. Non-patent document 3 shows that NAD+ decreases in the hippocampus of mouse brain with aging and such decrease is associated with hypersensitive behaviors to fear and anxiety, while administration of NMN may alleviate hypersensitive behaviors to fear and anxiety. Non-patent document 4 reports that administration of melatonin and NMN, each alone or in combination, to old rats improves their learning and memory abilities. Further, Non-patent document 5 shows that administration of NMN to a mouse model of drug-induced depression improves drug-induced depression-like symptoms and such improvement is associated with mitochondrial energy metabolism in the brain. Patent document 1 describes an Alzheimer's disease-like symptom alleviating agent characterized by using one selected from R-NMN, a pharmacologically acceptable salt thereof, and a solvate of them as an active ingredient. This reference states that the results of experiments in mouse models of Alzheimer's disease suggest that R-NMN may be able to prevent amyloid-s-induced impairments of spatial cognition and impairments of learning and memory abilities.

By the way, addition of NMN to foods and drinks for babies has been examined. For example, Patent document 2 describes a powdered milk composition for infants containing at least one kind of nicotinyl riboside compound selected from the group consisting of nicotinamide riboside (NR, I), nicotinic acid riboside (NAR, II), nicotinamide mononucleotide (NMN, III), reduced nicotinamide riboside (NRH, IV), reduced nicotinic acid riboside (NARH, V), nicotinamide riboside triacetate (NRTA, VI), nicotinic acid riboside triacetate (NARTA, VII), reduced nicotinamide riboside triacetate (NRH-TA, VIII), and reduced nicotinic acid riboside triacetate (NARH-TA, IX). This reference also describes that feeding nicotinamide riboside to piglets, a model of infant intestinal development, resulted in increased levels of short-chain fatty acids in feces. Further, Patent document 3 describes a food or drink for babies and infants characterized by containing an effective amount of NMN. This reference also describes that IgM and IgG levels and CD8-positive T-cell count increased in blood of babies aged 1.8 to 6.5 years who continuously consumed NMN-added powdered milk formula for 30 days.

Furthermore, the effect of duration of breastfeeding on children's intelligence has been reported (Non-patent document 6). It has also been reported that mice raised by mothers supplemented with nicotinamide riboside (NR) tend to have lower fat and larger brains at weaning, NR stimulates milk secretion and the production of brain-derived neurotrophic factor (BDNF), and NR supplementation results in twice as high BDNF level in breast milk, suggesting a powerful mechanism by which NR supplementation supports the development and health of newborns (Patent document 4 and Non-patent document 7).

### PRIOR ART REFERENCES

### Patent Documents

Patent document 1: Japanese Patent Publication (Kokai) No. 2022-080787
Patent document 2: Japanese Patent Publication (Kohyo) No. 2019-513385
Patent document 3: International Publication WO2020/166527
Patent document 4: International Publication WO2018/102426 (Japanese Patent Publication (Kohyo) No. 2020-504168, Japanese Patent No. 7136795)

### Non-Patent Documents

Non-patent document 1: Redox Biol. 2019 June; 24:101192
Non-patent document 2: Geroscience. 2020 April; 42(2):527-546
Non-patent document 3: NPJ Aging Mech Dis. 2018 Nov. 8; 4:10
Non-patent document 4: Neuroscience. 2019 Dec. 15; 423:29-37
Non-patent document 5: J Affect Disord. 2020 Feb. 15; 263:166-174
Non-patent document 6: Breastfeed Med. 2021: https://doi.org/10.1089/bfm.2020.0364
Non-patent document 7: Cell Rep. 2019 Jan. 22; 26(4):969-983.e4

### SUMMARY OF THE INVENTION

### Problem to be Solved by the Invention

Although NMN has been reported to act as an anti-aging agent, the effect of NMN contained in breast milk on the development of brain functions in infants has been unknown.

### Means for Solving the Problem

The present invention provides the followings.
[1]A composition for promoting development of a brain function in an infant or child, the composition comprising a nicotinamide adenine dinucleotide (NAD)-related substance or a salt thereof acceptable for use in foods or pharmaceuticals.
[2] The composition according to 1, wherein the development of a brain function is development in any area selected from the group consisting those of communication, gross motor, fine motor, problem solving, and personal-social.
[3] The composition according to 1 or 2, wherein the development of a brain function is development in any area selected from the group consisting those of fine motor, problem solving, and personal-social.
[4] The composition according to any one of 1 to 3, wherein the development of a brain function is development in the area of personal-social.
[5] The composition according to any one of 1 to 4, wherein the development of a brain function is development of a brain function in a child at the age of 23 months or older.
[6] The composition according to any one of 1 to 5, which is for ingestion by an infant at the age of 6 months or younger.
[7] The composition according to any one of 1 to 6, which is in the form of any selected from the group consisting of formulated milk powder, formulated milk liquid, and breast-milk substitute.
[8] The composition according to any one of 1 to 7, wherein concentration of the NAD-related substance or a salt thereof acceptable for use in foods or pharmaceuticals at the time of ingestion is 1.9 μg/mL or higher in terms of nicotinamide mononucleotide (NMN) concentration.
[9] The composition according to any one of 1 to 8, wherein concentration of the NAD-related substance or a salt thereof acceptable for use in foods or pharmaceuticals at the time of ingestion is 3.0 μg/mL or higher in terms of NMN concentration.
[10] The composition according to any one of 1 to 9, which is in the form of formulated milk powder and comprises 1.1 mg or more of the NAD-related substance or a salt thereof acceptable for use in foods or pharmaceuticals in terms of amount of NMN per 100 g of the composition.
[11] The composition according to any one of 1 to 10, which is in the form of formulated milk powder and comprises 1.8 mg or more of the NAD-related substance or a salt thereof acceptable for use in foods or pharmaceuticals in terms of amount of NMN per 100 g of the composition.
[12] The composition according to any one of 1 to 11, wherein the NAD-related substance is NMN.
[13]A method for promoting development of a brain function in an infant or child, the method comprising the steps of producing an artificial nutritional composition comprising an NAD-related substance or a salt thereof acceptable for use in foods or pharmaceuticals, and making an infant or child in need of promoting development of the brain function ingest the produced composition: a non-therapeutic method for promoting development of a brain function in an infant or child, the method comprising the step of making an infant or child for whom development of the brain function is desirable ingest a composition comprising an NAD-related substance or a salt thereof acceptable for use in foods or pharmaceuticals; a composition for use in a method for promoting development of a brain function in an infant or child, the composition comprising an NAD-related substance or a salt thereof acceptable for use in foods or pharmaceuticals: use of a composition comprising an NAD-related substance or a salt thereof acceptable for use in foods or pharmaceuticals in manufacture of a composition for promoting development of a brain function in an infant or child; or use of a composition comprising an NAD-related substance or a salt thereof acceptable for use in foods or pharmaceuticals for promoting development of a brain function in an infant or child.
[14] The method, composition, or use according to 13, wherein the development of a brain function is development in any area selected from the group consisting of those of communication, gross motor, fine motor, problem solving, and personal-social.
[15] The method, composition, or use according to 13 or 14, wherein the development of a brain function is development in any area selected from the group consisting of those of fine motor, problem solving, and personal-social.
[16] The method, composition, or use according to any one of 13 to 15, wherein the development of a brain function is development in the area of personal-social.
[17] The method, composition, or use according to any one of 13 to 16, wherein the development of a brain function is development of a brain function in a child at the age of 23 months or older.
[18] The method, composition, or use according to any one of 13 to 17, wherein the composition is for ingestion by an infant at the age of 6 months or younger.
[19] The method, composition, or use according to any one of 13 to 18, wherein the composition is in the form of any selected from the group consisting of formulated milk powder, formulated milk liquid, and breast-milk substitute.
[20] The method, composition, or use according to any one of 13 to 19, wherein concentration of the NAD-related substance or a salt thereof acceptable for use in foods or pharmaceuticals at the time of ingestion is 1.9 μg/mL or higher in terms of NMN concentration.
[21] The method, composition, or use according to any one of 13 to 20, wherein concentration of the NAD-related substance or a salt thereof acceptable for use in foods or pharmaceuticals at the time of ingestion is 3.0 μg/mL or higher in terms of NMN concentration.
[22] The method, composition, or use according to any one of 13 to 21, wherein the composition is in the form of formulated milk powder and comprises 1.1 mg or more of the NAD-related substance or a salt thereof acceptable for use in foods or pharmaceuticals in terms of amount of NMN per 100 g of the composition.
[23] The composition according to any one of 13 to 22, wherein the composition is in the form of formulated milk powder and comprises 1.8 mg or more of the NAD-related substance or a salt thereof acceptable for use in foods or pharmaceuticals in terms of amount of NMN per 100 g of the composition.
[24] The composition according to any one of 13 to 23, wherein the NAD-related substance is NMN.

The present invention also provides the followings.
[31] A composition for promoting development of a brain function in an infant or child, the composition comprising nicotinamide mononucleotide (NMN).
[32] The composition as according to 31, wherein the development of a brain function is development in any area selected from the group consisting of those of communication, gross motor, fine motor, problem solving, and personal-social.
[33] The composition according to 31 or 32, wherein the development of a brain function is development in any area selected from the group consisting of those of fine motor, problem solving, and personal-social.
[34] The composition according to any one of 31 to 33, wherein the development of a brain function is development in the area of personal-social.
[35] The composition according to any one of 31 to 34, wherein the development of a brain function is development of a brain function in a child at the age of 23 months or older.
[36] The composition according to any one of 31 to 35, which is for ingestion by an infant at the age of 6 months or younger.
[37] The composition according to any one of 31 to 36, which is in the form of any selected from the group consisting of formulated milk powder, formulated milk liquid, and breast-milk substitute.
[38] The composition according to any one of 31 to 37, wherein concentration of NMN at the time of ingestion is 1.9 μg/mL or higher.
[39] The composition according to any one of 31 to 38, wherein concentration of NMN at the time of ingestion is 3.0 μg/mL or higher.
[40] The composition according to any one of 31 to 39, which is in the form of formulated milk powder and comprises 1.1 mg or more of NMN per 100 g of the composition.
[41] The composition according to any one of 31 to 40, which is in the form of formulated milk powder and comprises 1.8 mg or more of NMN per 100 g of the composition.
[42] A method for promoting development of a brain function in an infant or child, the method comprising the steps of producing an artificial nutritional composition comprising NMN; and making an infant or child in need of promoting development of the brain function ingest the produced composition; a non-therapeutic method for promoting development of a brain function in an infant or child, the method comprising the step of making an infant or child for whom development of the brain function is desirable ingest a composition comprising NMN; a composition for use in a method for promoting development of a brain function in an infant or child, the composition comprising NMN; use of a composition comprising NMN in manufacture of a composition for promoting development of a brain function in an infant or child; or use of a composition comprising NMN for promoting development of a brain function in an infant or child.
[43] The method, composition, or use according to 42, wherein the development of a brain function is development in any area selected from the group consisting of those of communication, gross motor, fine motor, problem solving, and personal-social.
[44] The method, composition, or use according to 42 or 43, wherein the development of a brain function is development in any area selected from the group consisting of those of fine motor, problem solving, and personal-social.
[45] The method, composition, or use according to any one of 42 to 44, wherein the development of a brain function is development in the area of personal-social.
[46] The method, composition, or use according to any one of 42 to 45, wherein the development of a brain function is development of a brain function in a child at the age of 23 months or older.
[47] The method, composition, or use according to any one of 42 to 46, wherein the composition is for ingestion by an infant at the age of 6 months or younger.
[48] The method, composition, or use according to any one of 42 to 47, wherein the composition is in the form of any selected from the group consisting of formulated milk powder, formulated milk liquid, and breast-milk substitute.
[49] The method, composition, or use according to any one of 42 to 48, wherein concentration of NMN at the time of ingestion is 1.9 μg/mL or higher.
[50] The method, composition, or use according to any one of 42 to 48, wherein the concentration of NMN at the time of ingestion is 3.0 μg/mL or higher.
[51] The method, composition, or use according to any one of 42 to 48, wherein the composition is in the form of formulated milk powder and comprises 1.1 mg or more of NMN per 100 g of the composition.
[52] The composition according to any one of 42 to 48, which is in the form of formulated milk powder and comprises 1.8 mg or more of NMN per 100 g of the composition.

### Effects of the Invention

The composition of the present invention can contribute to future healthy growth of infants or children by promoting development of their brain functions.

Since NMN, which is used as an active ingredient of the composition of the present invention, is originally contained in breast milk, the composition of the present invention can safely exhibit the desired effect in infants or children.

There can be provided a composition that promotes development of a brain function in the same way as breastfeeding even when breastfeeding is not available.

The present invention provides information on the relationship between NMN ingested during infancy and development of brain function.

The composition of the present invention is expected to be applicable to the treatment of diseases or conditions relating to brain function in infants or children. The composition of the present invention is also expected to be applied to promotion of development of brain functions, improvement of nutritional conditions, and maintenance and promotion of health not only in infants but also in young children, children, adults, and elderly.

### BRIEF DESCRIPTION OF THE DRAWINGS

FIG. 1 Comparison between groups divided with a boundary value of 1.922 μg/ml. Mean±standard error, *p<0.05 by Mann-Whitney U, n=22 for low NMN, and n=128 for high NMN.

FIG. 2 Comparison between groups divided with a boundary value of 3.089 μg/ml. Mean±SEM, *p<0.05 by Mann-Whitney U, n=76 for low NMN, and n=74 for high NMN.

FIG. 3 Distributions of NMN, NAD, NR, and NA concentrations in 1-month breast milk. The horizontal lines showing the distributions of NMN, NAD, NR, and NA concentrations in the figure indicate, from the lower concentrations, the minimum value (0.058 μg/ml for NMN, 0.154 μg/ml for NAD, 0.006 μg/ml for NR, and 0.000 μg/ml for NA), first quartile (2.352 μg/ml for NMN, 1.069 μg/ml for NAD, 0.050 μg/ml for NR, and 0.000 μg/ml for NA), second quartile (median, 3.075 μg/ml for NMN, 1.481 μg/ml for NAD, 0.119 μg/ml for NR, and 0.000 μg/ml for NA), third quartile (3.758 μg/ml for NMN, 1.958 μg/ml for NAD, 0.422 μg/ml for NR, and 0.002 μg/ml for NA), and maximum value (8.043 μg/ml for NMN, 4.657 μg/ml for NAD, 1.532 μg/ml for NR, and 0.002 μg/ml for NA).

### MODES FOR CARRYING OUT THE INVENTION

### [Active Ingredient]

The composition of the present invention comprises a nicotinamide adenine dinucleotide (NAD)-related substance, such as nicotinamide mononucleotide (NMN), as the active ingredient.

There are two types of optical isomers of NMN, α-type and β-type, but in the present invention, NMN refers to β-type NMN (β-nicotinamide mononucleotide), unless especially stated. NMN is an intermediate metabolite of NAD+. The term active ingredient refers to an ingredient that contributes to the intended purpose, and in the case of food with a functional claim, it is sometimes referred to as a functional substance.

In one embodiment, NMN may be contained in the composition as a salt thereof acceptable for use in foods or pharmaceuticals. For the present invention, the expression that the composition comprises NMN also means a state that NMN is contained in the form of a salt thereof acceptable for use in foods or pharmaceuticals.

The salt acceptable for use in foods or pharmaceuticals refers to any salt selected from the group consisting of nitrate, sulfate, carbonate, hydrogencarbonate, halide, formate, acetate, citrate, tartrate, oxalate, fumarate, salt of a saturated or unsaturated fatty acid with 3 to 20 carbons, salt of carnitine or a derivative thereof, salt of hydroxycitric acid or a derivative thereof, salt of ascorbic acid or a derivative thereof, salt of ascorbyl phosphate or a derivative thereof, sodium salt, potassium salt, calcium salt, magnesium salt, zinc salt, and ammonium salt.

In one embodiment, NMN may be contained in the composition as an NAD-related substance or a salt thereof acceptable for use in foods or pharmaceuticals. For the present invention, the expression that the composition comprises NMN also means a state that NMN is contained in the form of an NAD-related substance or a salt thereof acceptable for use in foods or pharmaceuticals.

The term NAD-related substance refers to NAD or a substance that can be converted into NAD in vivo. NAD functions as a coenzyme for various dehydrogenases in vivo and can take two states, oxidized type (NAD⁺) and reduced type (NADH). The NAD-related substance may be NMN, nicotinamide riboside (NR), NAD, nicotinamide (NAM, also called niacinamide), nicotinic acid (NA), NaAD (nicotinic acid adenine dinucleotide), or NaMN (nicotinic acid mononucleotide). For the present invention, NAD may be either oxidized type NAD⁺ or reduced type NADH, unless especially noted.

The NAD-related substance may be a nicotinamide mononucleotide derivative, which is a compound represented by the general formula (1).

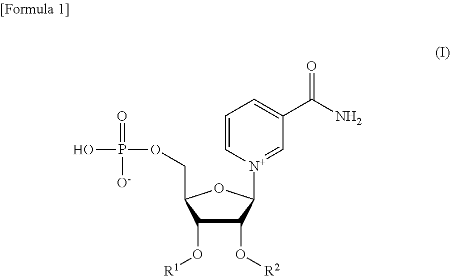

In the formula, R¹ and R² are independently an acyl group having 6 to 16 carbons, and the hydrocarbon group attached to the carbonyl carbon of the acyl group is a linear or branched-chain saturated or unsaturated hydrocarbon group. Such a compound can be produced by an acylation reaction of NMN with an acylation agent selected from the group consisting of a carboxylic acid having an acyl group of 6 to 16 carbons in which a linear or branched-chain saturated or unsaturated hydrocarbon group attaches to the carbonyl carbon, a halide of such a carboxylic acid, and an anhydride of such a carboxylic acid in a solvent containing at least 20% by mass of a strongly acidic liquid having a pKa of 2.0 or lower (see WO2017/110317).

In a preferred embodiment, the composition comprises, as the active ingredient, any selected from NMN, NAD, NR, NA, NAM, NaAD, and NaMN, preferably any selected from NMN, NAD, NR, and NA, more preferably NMN.

NMN, NAD-related substance, and a salt thereof acceptable for use in foods or pharmaceuticals can be produced by various methods. They may be synthesized or extracted from natural products or cultures of yeast etc. containing the substances.

### Use

### Function and Action

The composition of the present invention can be used to promote development of a brain function in infants or children. The development of a brain function includes those that can be assessed by various existing tests. Examples of the existing tests include developmental screening (questionnaires), such as Ages & Stages Questionnaires (registered trademark). Third Edition (ASQ (registered trademark)-3) and translated versions thereof, the Enjohji Developmental Test in infancy and Early Childhood, the Denver Developmental Screening Test (DDST) and Japanese version thereof (JDDST); and individual developmental tests, such as the new version of the K-type developmental test.

In a preferred embodiment, the composition is used to promote development in any of five developmental areas. The five developmental areas mean developmental areas of communication (babbling, vocalization, listening and understanding), gross motor (arm, leg, and whole body movements), fine motor (hand and finger movements), problem solving (learning and playing with toys), and personal-social (playing alone and with toys and other children that demonstrate social skills). For example, promotion of development in the developmental area of communication is specifically indicated in the form of promotion of babbling and vocalization and improvement in comprehension through listening. Therefore, in a more specific embodiment, the composition is used for promotion of any ability selected from the group consisting of abilities of babbling, vocalization, listening and understanding through listening; for promotion of any motor ability selected from the group consisting of motor abilities of arm, leg, and whole body; for promotion of hand and finger movements; for promotion of learning ability; and for promotion of social skills.

In one embodiment, the composition is used for promotion of the development in any developmental area selected from the group consisting of the developmental areas of communication, gross motor, fine motor, problem solving, and personal-social, preferably for promotion of the development in all of these areas. In another embodiment, the composition is used for promotion of the development in, among the five areas, any of areas including at least the personal-social area. In another embodiment, the composition is used for promotion of the development in any area selected from the group consisting of the areas of fine motor, problem solving, and personal-social, preferably for promotion of the development in all of the areas of fine motor, problem solving, and personal-social.

The development in the five areas can be assessed with Ages & Stages Questionnaires (registered trademark), Third Edition (ASQ (registered trademark)-3) or the Japanese version thereof. When using the ASQ (registered trademark)-3, the questionnaire should be selected according to the exact monthly or yearly age of the subject infant or child. The Japanese versions of the questionnaire are available for ages of 6, 12, 18, 24, 30, 36, 42, 48, 54, and 60 months. If the subject infant or child was born before 37 weeks and 0 day of gestation, and is younger than 2 years old at the time of evaluation, the questionnaire should be selected on the basis of the modified age in months. The questionnaire is usually completed by the parent or caregiver. The completed scores are totalized, scores are corrected if necessary, and the developmental status of each area can be assessed by using a graph showing cutoff value for each area.

In one embodiment, the composition is used for promotion of development of a brain function in children at the age of 23 months or older. According to the studies of the inventors of the present invention, a high correlation was observed between the amount of NMN in breast milk ingested from 1 to 6 months of age and development assessed by using the Japanese version of the ASQ-3 24-month Information Summary (target age in months is 23 months 0 days to 25 months 15 days).

The expression that development of a brain function is promoted by a certain composition means that the composition provides faster development in any of the five areas (higher scores are calculated on the ASQ (registered trademark)-3) than when a composition comprising no NMN or a lower concentration of NMN is fed, as well as the composition helps smooth development in any of the five areas, brings development in any of the five areas closer to a level of smooth development when the level of the development is within such a range that observation is required, and so forth.

The composition of the present invention may also be used for the treatment of a disease or condition relating to a brain function that may be expected to be improved by promoting development of the brain function. Examples of such a disease or condition include developmental disorders. Developmental disorders are disorders relating to the development of brain function. Developmental disorders include autism, Asperger's syndrome and other pervasive developmental disorders, learning disabilities, attention deficit hyperactivity disorder, Tourette syndrome, and stuttering.

The composition of the present invention can also be used to maintain healthy brain function, to maintain good brain function development, and to help them.

For the present invention, treatment of a disease or condition includes reducing the risk of developing the disease, delaying the onset of the disease, preventing, curing the disease, arresting and delaying the progression of the disease. The treatment includes medical treatment performed by a physician for the purpose of treating a disease, and non-medical treatment performed by a person other than a physician, such as a nutritionist, registered dietitian, public health nurse, midwife, nurse, clinical laboratory technician, food manufacturer, food seller, etc. The treatment also includes recommendation for the administration or intake of a specific food, dietary guidance, health guidance, nutritional guidance (including guidance on nutrition necessary for the medical treatment of injured and sick persons and nutritional guidance for maintenance and promotion of health), food service management, and guidance relating to food service necessary for nutritional improvement. Subjects of the treatment according to the present invention include humans (individuals) and non-human mammalian animals (such as companion animals).

### Subject

The composition of the present invention is suitable for ingestion by subjects for whom promotion of development of a brain function is desirable or necessary. Such subjects include infants (younger than 1 year old, including newborns up to 1 month old), young children (1 year old or older, but younger than 6 years old), children (6 years old or older, but younger than 15 years old), adults (15 years old or older), middle-aged and older persons (40 years old or older, but younger than 65 years old), elderly persons (65 years old or older), pregnant women, maternity women, persons in or after sickness, men and women.

In one embodiment, the composition is used for ingestion by infants or children (infants, young children, and children), preferably infants or young children, more preferably infants (e.g., 6 months old or younger). In another embodiment, the composition is suitable for ingestion by subjects for whom artificial nutrition is desirable instead of breastfeeding, or who are in need of artificial nutrition.

### Composition

### (Dosage Form or Shape)

The composition of the present invention can be prepared in any shape, such as solid, liquid, mixture, suspension, emulsion, gel, and sol. The composition of the present invention can be a synthesized nutritional composition prepared by mixing various ingredients.

In a particularly preferred embodiment, the composition of the present invention is in the form of formulated milk powder, formulated milk liquid, or breast-milk substitute. Formulated milk powder means the product which is obtained from product made by processing food made from raw milk, cow's milk or special cow's milk, or made from them as principal raw materials, by adding the necessary nutrients for infants and young children, and then reducing to powder.
Formulated milk liquid means the product which is obtained from product made by processing food made from raw milk, cow's milk or special cow's milk, or made from them as principal raw materials, by adding the necessary nutrients for infants and young children, and then making it into a liquid.

The formulated milk powder, formulated milk liquid, or breast-milk substitute can be for infants, follow-up, low-birth weight infants, children, adults, elderly persons, or persons with allergy, lactose intolerance, or congenital metabolic disorders. The term for follow-up means for ingestion of well-balanced nutrients needed during infancy and early childhood, from about 9 months old after the weaning period to about 3 years old.

In one embodiment, the formulated milk powder is dissolved at a concentration of 5 to 17%, preferably 8 to 16%, more preferably 12 to 15%, at the time of use. The formulated milk powder can be in the form of stick or cube for a single serving (e.g., amount for dissolving it in hot water to prepare 200 ml of milk).

The composition of the present invention can be a composition that is a food or drug, other than formulated milk powder, formulated milk liquid, or breast-milk substitute. The food and drug include those for animals other than humans as well as those for humans, unless especially noted. The food may be a common food, functional food, food for young children or nutritional composition, or may be a therapeutic diet (diet for the purpose of therapy, for which a medical practitioner writes a dietary prescription, and which is cooked in accordance with a menu prepared by a dietitian or the like according to the prescription), dietetic food, ingredient-modified food, care food, or therapy-supporting food, unless especially indicated. The food is not limited to a solid food, but it may be a food in the form of liquid, for example, drink, drinkable preparation, liquid food, or soup, unless especially indicated. The term functional food refers to a food that can give a predetermined functionality to a living body, and includes health foods at large, such as foods for specified health uses (abbreviated as “Tokuho” in Japanese, including conditional foods for specified health use), foods with function claims, foods with health claims including foods with nutrient function claims, foods for special dietary uses, supplements (for example, those of various kinds of dosage forms such as tablet, coated tablet, sugar-coated tablet, capsule and solution), and cosmetic food (for example, diet foods). In the present invention, the “functional foods” include health foods to which the health claim based on the food standards of CODEX (JOINT FAO/WHO FOOD STANDARDS PROGRAMME CODEX ALIMENTARIUS COMMISSION) is applied. Cosmetics include medicinal cosmetics.

When the composition is a food, it can be in any form, such as supplement, confectionery, drink, drinkable preparation, seasoning, dairy product, processed food, daily dish, and soup. Specifically, the composition of the present invention can be in the form of gummy, tablet, drink, fermented milk, dairy drink, dairy beverage, soft drink, ice cream, cheese, bread, biscuit, cracker, pizza crust, formulated milk powder, liquid diet, food for sick persons, nutritional food, frozen food, processed food, or the like, or may be in the form of granule, powder, paste, thick solution or the like for being mixed in drink or food or diluted to prepare a drink or the like. Specifically, the composition may be in the form of syrup for adding to milk.

When the composition is a drug, specific examples of the form thereof include chewable tablet, sublingual tablet, pill, capsule, granule, powder, liquid, suspension, emulsion, syrup, liniment, lotion, gel, aerosol, spray, ointment, cream, tape, poultice, eye drop, nasal drop, and suppository.

### (Content and Dose of NMN)

The NMN content and concentration in the composition may be such an amount or concentration that the desired effect can be exhibited, and can be appropriately set in consideration of various factors such as target's age in months, age in years, weight, and desired effect. For the present invention, when the amount or concentration of NMN or the like in the composition is indicated, it means the amount or concentration as NMN, unless especially stated. In other words, the amount or concentration of an NAD-related substance or a salt thereof acceptable for use in foods or pharmaceuticals in a composition indicated for the present invention means the amount or concentration thereof in terms of amount of NMN, unless especially stated. That is, when a salt of NMN acceptable for use in foods or pharmaceuticals or an NAD-related substance other than NMN is used as the active ingredient instead of NMN, the salt of NMN acceptable for use in foods or pharmaceuticals or NAD-related substance may be used in the same molar amount or molar concentration as that of NMN in the indicated amount. This conversion of amount or concentration can be easily performed by those skilled in the art.

When the composition is a liquid, e.g., formulated milk liquid, the concentration of NMN in the composition is preferably 0.05 μg/mL or higher, more preferably 1.0 μg/mL or higher, from the viewpoint of promoting the development in any of the five areas mentioned above. From the viewpoint of more clearly promoting the development in any of the five regions mentioned above including at least the area of personal-social, the concentration of NMN is preferably 1.9 μg/mL or higher, more preferably 2.3 μg/mL or higher, further preferably 2.5 μg/mL or higher, still further preferably 3.0 μg/mL or higher. From the viewpoint of more clearly promoting the development in any area selected from the group consisting of those of fine motor, problem solving, and personal-social, preferably in all areas of fine motor, problem solving, and personal-social, the concentration of NMN is preferably 3.0 μg/mL or higher, and may be 3.5 μg/mL or higher, or may be 3.7 μg/mL.

When the composition is a liquid, e.g., formulated milk liquid, the maximum concentration of the NMN in the composition can be appropriately set, for example, it may be 50 μg/mL or lower, 40 μg/mL or lower, 30 μg/mL or lower, 20 μg/mL or lower, or 10 μg/mL or lower. From the viewpoint that the concentration is preferably within the range of the amount of NMN normally contained in breast milk, the concentration of NMN is preferably 8.1 μg/mL or lower, and may be 6.0 μg/mL or lower.

When the composition is a solid, e.g., formulated milk powder, the amount of NMN per 100 g of the composition should is preferably 0.03 mg or more, more preferably 1.0 mg or more, from the viewpoint of promoting the development in any of the five areas mentioned above. From the viewpoint of more clearly promoting the development in any of the five areas described above including at least the personal-social area, the amount of NMN per 100 g of the composition is preferably 1.1 mg or more, more preferably 1.3 mg or more, further preferably 1.4 mg or more, still further preferably 1.8 mg or more. From the viewpoint of more clearly promoting the development in any area selected from the group consisting of those of fine motor, problem solving, and personal-social, preferably in all areas of fine motor, problem solving, and personal-social, the amount of NMN per 100 g of the composition is preferably 1.8 mg or more, and may be 2.0 mg or more or 2.2 mg or more.

When the composition is a solid, e.g., formulated milk powder, the maximum amount of NMN per 100 g of the composition may be appropriately set, and it can be, for example, 60 mg or less, or may be 50 mg or less, 40 mg or less, 30 mg or less, or 20 mg or less. From the viewpoint that the amount is preferably within the range of the amount normally contained in breast milk, the amount of NMN per 100 g of the composition is preferably 16.1 mg or less, and may be 13 mg or less.

The content of NMN in the composition may be designed taking the amount of NMN to be ingested per day into account. The daily dose of NMN may be 0.50 mg or more, or 1.0 mg or more. From the viewpoint of more clearly promoting the development in any area selected from the group consisting of those of fine motor, problem solving, and personal-social, the daily dose of NMN is preferably 1.9 mg or more, more preferably 2.5 mg or more. From the viewpoint of more clearly promoting the development in the area of personal-social, the daily dose of NMN is preferably 3.0 mg or more, more preferably 6.0 mg or more.

The maximum daily dose of NMN can be appropriately set, and it can be, for example, 50 mg or less, and may be 40 mg or less, 30 mg or less, 20 mg or less, or 10 mg or less. From the viewpoint of considering the range of the amount normally contained in breast milk, the daily dose of NMN is preferably be 8.1 mg or less, and may be 6.0 mg or less. Such a daily dose may be divided into multiple portions, e.g., 3 to 7 portions, so as to be suitable for ingestion of 3 to 7 times per day.

The composition may be ingested at any time point in the day. If the composition is formulated milk powder, formulated milk liquid, or breast-milk substitute, the composition of the present invention may be used for milk feeding in the same manner as in the case of breastfeeding.

In view of the fact that the NAD concentration in the body to be used is considered to fluctuate during the day, and that it increases during the active period, in one embodiment, it is considered to be preferable that the composition be ingested more in the morning, corresponding to the beginning of the active period.

### (Other Ingredients and Additives)

In addition to NMN, the composition of the present invention may comprise other nutritional ingredients that are acceptable for use in foods or pharmaceuticals. For example, if the composition is in the form of formulated milk powder, formulated milk liquid, or breast-milk substitute, it may comprise, as ingredients other than NMN, proteins, fats, cholesterols, carbohydrates, niacin, pantothenic acid, biotin, vitamin A, vitamin B1, vitamin B2, vitamin B6, vitamin B12, vitamin C, vitamin D, vitamin E, vitamin K, folic acid, zinc, sodium, potassium, calcium, selenium, iron, copper, magnesium, phosphorus, lactadherin, α-lactalbumin, β-lactoglobulin, ARA (arachidonic acid), DHA (docosahexaenoic acid), linoleic acid, α-linolenic acid, phospholipids, fructooligosaccharides, inositol, β-carotene, chlorine, carnitine, taurine, nucleotides, etc.

The composition may also comprise other active ingredients or nutritional ingredients acceptable for use in foods or pharmaceuticals, in addition to NMN.

The composition may further comprise additives that are acceptable for use in foods or pharmaceuticals. Examples of such additives include inert carriers (solid or liquid carriers), excipients, surfactants, binders, disintegrants, lubricants, dissolution aids, suspending agents, coating agents, colorants, preservatives, buffering agents, pH adjusters, emulsifiers, stabilizers, sweeteners, antioxidants, flavorings, acidifiers, and natural products.

### (Administration)

The composition of the present invention is orally used. The composition may also be administered parenterally, e.g., transluminally (gastrostomy, enterostomy etc.) or intranasally, but it is desirable to use it orally.

The composition can be repeatedly ingested by the subject and can be continuously ingested by the subject over a long period of time. The period is not particularly limited, but in order to fully recognize the effect, it is preferable to have the subject take the composition continuously for a relatively long period of time, for example, 1 week or longer, 2 weeks or longer, 1 month or longer, 3 months or longer, 6 months or longer, or 1 year or longer. Since the active ingredient, NMN, has long been taken as an ingredient of foods and NMN is originally contained in breast milk, the composition of the present invention is particularly suitable for long-term ingestion.

### Others

The composition of the present invention may be used in conjunction with ingestion of other ingredients, ingestion of health foods or supplements, exercise, normal diet, and so forth. The composition of the present invention may also be used in combination with a therapy that promotes development of a brain function. Examples of such a therapy that promotes development of a brain function include inclusive therapy, music therapy, occupational therapy, social skills training, cognitive behavioral therapy, and therapeutic education method.

The composition of the present invention can be appropriately manufactured by those skilled in the art using existing facilities and other equipments. In the manufacture of the composition, the stage of blending of NMN is not particularly limited as long as the properties of NMN are not significantly impaired.

The composition of the present invention can be produced according to the usual methods, even when it is in the form of formulated milk powder, formulated milk liquid, or breast-milk substitute. For example, when it is in the form of formulated milk powder, it can be manufactured as follows. Processed whey proteins, casein, processed soy proteins, sugar (lactose, oligosaccharide etc.), minerals, vitamins, fats and oils (containing ARA, DHA, linoleic acid, (X-linolenic acid, etc. as fatty acid) can be blended, dissolved, mixed, cleaned, then sterilized, concentrated, and homogenized, and the resulting product can be subjected to a drying process such as spray drying to obtain formulated milk powder. Also in this case, the stage of adding NMN is not particularly limited, as long as the characteristics of NMN are not significantly impaired.

Products comprising the composition of the present invention can have an indication of the function and purpose of use (intended use) thereof, and can also have an indication of a recommendation for administration or ingestion thereof to or by a specific target. The indication may be a direct or indirect indication. Examples of the direct indication include descriptions on tangible articles such as the product itself, package, container, label, and tag, and examples of the indirect indication includes advertising and campaign activities using such places or means as web site, shop, pamphlet, exhibition, seminar such as media seminar, book, newspaper, magazine, television, radio, postal matter, E-mail, and sound. Examples of the function and purpose of use (intended use) to be indicated include “to help development of brain functions”, “to help maintenance of brain functions”. “for babies of 6 months old or younger”, “for infants”, “for young children”, etc.

Hereafter, the present invention will be more specifically explained with reference to the following examples.

### EXAMPLES

### [Effect of NMN to Promote Development in Infants]

### Method 1

The effect of the amount of nicotinamide adenine dinucleotide (NAD)-related substances ingested by infants on their future brain function development was evaluated. The method was specifically as follows.

Concentrations of NAD-related substances in 1-month breast milk ingested by 150 infants of 1 to 6 months old, who were fully breastfed, were determined by LC-MS/MS. As the NAD-related substances, NAD, nicotinamide riboside (NR), nicotinic acid (NA), nicotinamide (NAM), and nicotinamide mononucleotide (NMN) were evaluated.

The measurement of the NAD-related substances in breast milk was performed as follows. Cryopreserved breast milk was thawed and sonicated at 40° C. for 10 minutes. To 40 μL of the breast milk, 1460 μL of 82.2% (v/v) methanol was added, and the mixture was stirred and then sonicated for 30 seconds. An internal standard solution (80 ng/mL β-nicotinamide-d4 mononucleotide solution prepared with 80.0% (v/v) methanol solution) was added in a volume of 100 μL, and the resulting mixture was stirred and then centrifuged at 17,900 g and 4° C. for 10 minutes. The supernatant (50 μL) was concentrated by centrifugation, and re-dissolved in 525 μL of 0.1 mg/mL aqueous citric acid solution, the solution was filtered through a 0.22-μm filter, and the filtrate was analyzed by liquid chromatography-mass spectrometry (LC-MS/MS) under the following conditions to determine the NMN concentration.

### (LC-MS/MS Analysis Conditions)

Detector: Xevo TQ-XS (Waters)
HPLC system: System produced by Waters
Column: ACQUITY PREMIER HSS T3 1.8 μm, 2.1 mm×150 mm column
Mobile phase
   Solution A: 5 mM Aqueous ammonium formate solution
   Solution B: Acetonitrile solution
   Ratio of liquid A and liquid B: 100 v/v % Solution A and 0 v/v % solution B from the start of the apply of the mobile phase to 0 minute; 90 v/v % solution A and 10 v/v % solution B from 0 minute to 3.0 minutes; 90 v/v % solution A and 10 v/v % solution B from 3.0 minutes to 3.5 minutes; 10 v/v % solution A and 90 v/v % solution B from 3.5 minutes to 8.0 minutes; and 100 v/v % solution A and 0 v/v % solution B from 8.0 minutes to 11.5 minutes.
Column temperature: 45° C.
Flow rate: 0.3 mL/minute
Injection volume: 2 μL
Ionization: ESI positive mode
NMN m/z
   Precursor ion (Ql Mass, (Da)): 335.2
   Product ion (Q3 Mass, (Da)): 123.0

Development of the infants was assessed at the age of 24 months using ASQ (registered trademark)-3 (Japanese version of ASQ (registered trademark)-3 infant developmental screening questionnaires, Igaku Shoin, 2021). The relationship between the amount of each NAD-related substance ingested by the infants and their development was evaluated by using Spearman's rank-order correlation coefficient.

### Method 2

A study was conducted to show that future development of infants' brain function varies depending on the amount of NMN ingested by the infants. The method was specifically as follows.

By using the data on the contents of NMN in breast milk and the developmental scores of the infants shown in Method 1, the subject infants were divided into two groups according to the concentration of NMN in breast milk, and the groups were compared according to Mann-Whitney's U-test. In this study, the boundary concentration of the two groups was set to be 1.922 μg/mL (mean NMN concentration in 1-month breast milk−1×standard deviation) or 3.089 μg/mL (mean NMN concentration in 1-month breast milk). That is, the group comparison was performed between two groups of NMN concentrations in the consumed breast milk of 21.922 μg/mL (high NMN group) and <1.922 μg/mL (low NMN group), or two groups of the same of 3.089 μg/mL (high NMN group) and <3.089 μg/mL (low NMN group).

### Method 3

The maximum amount of NMN in the composition ingested by the infants was estimated. The specific method was as follows.

From the NMN contents in breast milk shown in Method 1, a box-and-whisker diagram showing the distribution of NMN concentrations in breast milk was prepared. The maximum NMN concentration in breast milk observed in the obtained distribution was estimated as the maximum amount of NMN in the composition ingested by the infants.

### Result 1

The results of evaluation of the correlations between the scores in the five developmental areas of ASQ3 (communication, gross motor, fine motor, problem solving, and social-individual) at the age of 24 months and the concentration of each NAD-related substance in breast milk are shown in Tables 1 and 2.

The concentrations of NMN in breast milk ingested by the infants showed a significant positive correlation with the scores for all five developmental areas at 24 months (p<0.05). For NAD-related substances in breast milk other than NMN, the developmental areas of the infants for which the scores showed a positive correlation with their concentrations in breast milk were problem solving for NR, gross motor for NA, and communication for NAD (p<0.05) (Table 1). In the correlations between NAD-related substance concentrations and the scores of the developmental areas where significant positive correlations were found, all the correlation coefficients (r) were more strongly correlated with NMN concentration in breast milk (Table 2).

These results indicate that among the NAD-related substances, NMN is the most effective in promoting brain development in infants.

TABLE 1
Correlation analysis of NAD-related substances and developmental areas based on Spearman's rank correlation coefficient

| | | Correlation with | | Correlation with | | Correlation with | | Correlation with | | Correlation with | |
|---|---|---|---|---|---|---|---|---|---|---|---|
| | Developmental | NMN concentration | | NAD concentration | | NR concentration | | NA concentration | | NAM concentration | |
| | index | r | p value | r | p value | r | p value | r | p value | r | p value |
| 24- | Communication | 0.199 | 0.015 | 0.198 | 0.015 | 0.017 | 0.837 | 0.025 | 0.760 | 0.016 | 0.847 |
| month | Gross motor | 0.186 | 0.022 | 0.077 | 0.350 | 0.082 | 0.318 | 0.095 | 0.248 | −0.126 | 0.125 |
| | Fine motor | 0.208 | 0.011 | 0.025 | 0.762 | 0.085 | 0.303 | 0.169 | 0.039 | −0.002 | 0.981 |
| | Problem solving | 0.288 | p < 0.001 | 0.056 | 0.495 | 0.177 | 0.030 | 0.147 | 0.072 | −0.077 | 0.351 |
| | Social-individual | 0.269 | 0.001 | 0.048 | 0.559 | 0.117 | 0.154 | 0.154 | 0.060 | −0.211 | 0.010 |

TABLE 2
Comparison of correlation coefficients between
NMN and developmental areas and between other
NAD-related substances and developmental areas

| | | Correlation | Correlation with other | |
|---|---|---|---|---|
| | | with NMN | NAD-related substance | |
| | | concentra- | r² (Other NAD-related | |
| | Developmental | tion | substances that showed | r¹/ |
| | Index | r¹ | positive correlation) | r² |
| 24- | Communication | 0.199 | 0.198 (NAD) | 1.01 |
| month | Gross motor | 0.186 | — | — |
| | Fine motor | 0.208 | 0.169 (NA) | 1.23 |
| | Problem solving | 0.288 | 0.177 (NR) | 1.63 |
| | Social- | 0.259 | — | — |
| | individual | | | |

### Result 2

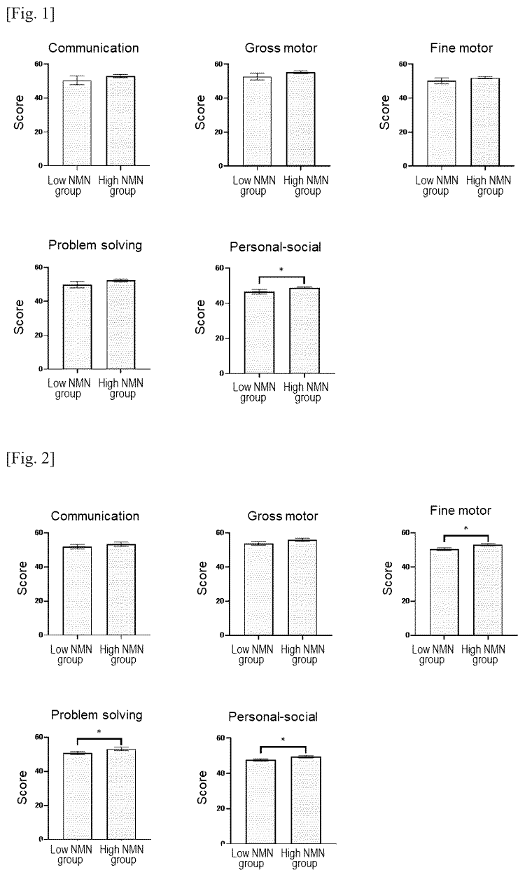

As a result of the between-group comparison, the development of the infants was significantly accelerated for the item of personal-social in the high NMN group in the case of the between-group comparison using the boundary value of 1.922 μg/mL (FIG. 1). In the between-group comparison using the boundary value of 3.089 μg/mL, the development of the infants was significantly accelerated for the items of fine motor, problem solving, and personal-social in the high NMN group (FIG. 2). These results indicate that infants' brain functions are significantly enhanced when the NMN level in the composition they drink is ≥1.922 μg/mL.

### Result 3

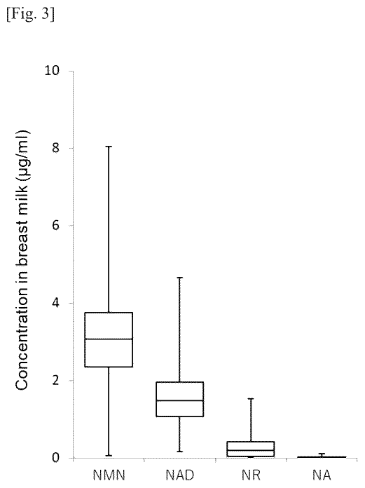

The distributions of NMN, NAD, NR, and NA concentrations in 1-month breast milk of the 150 samples are shown in FIG. 3. The maximum concentration was 8.043 μg/mL. From this result, it was confirmed that the maximum concentration of NMN for the ingestion by infants is preferably 8.043 μg/mL.

### Subsummary

(1) In this epidemiological study, the inventors of the present invention newly found that the concentrations of NAD-related substances in milk ingested by infants are associated with their brain function development.
(2) It was found that this brain function development promoting effect is effective when the concentration of NMN in the composition is 1.922 to 8.043 μg per ml (equivalent to 1.131 to 16.09 mg/100 g of the composition in the form of dry powder that is dissolved at a concentration of 5 to 17% (w/v)) at the time of ingestion.
(3) It was found that, more preferably, the effect is effective when the concentration of NMN in the composition is 3.089 to 8.043 μg per ml (equivalent to 1.817 to 16.09 mg/100 g of the composition in the form of dry powder that is dissolved at a concentration of 5 to 17% (w/v)) at the time of ingestion.

### INDUSTRIAL APPLICABILITY

According to the present invention, it is possible to provide pharmaceutical or food compositions that can promote development of brain functions in infants or children, and methods for manufacturing medicaments or food products. It is also expected that, for many infants or children, improvement of nutrition is realized, their healthy life is ensured, and their welfare is promoted by the present invention.

1-12. (canceled)

13. A method for promoting development of a brain function in an infant or child, the method comprising:
making an infant or child in need of promoting development of a brain function ingest the composition comprising a nicotinamide adenine dinucleotide (NAD)-related substance or a salt thereof acceptable for use in foods or pharmaceuticals.

14. The method according to claim 13, wherein the development of a brain function is development in any area selected from the group consisting those of communication, gross motor, fine motor, problem solving, and personal-social.

15. The method according to claim 13, wherein the development of a brain function is development in any area selected from the group consisting those of fine motor, problem solving, and personal-social.

16. The method according to claim 13, wherein the development of a brain function is development in the area of personal-social.

17. The method according to claim 13, wherein the development of a brain function is development of a brain function in a child at the age of 23 months or older.

18. The method according to claim 13, wherein the composition is for ingestion by an infant at the age of 6 months or younger.

19. The method according to claim 13, wherein the composition is in the form of any selected from the group consisting of formulated milk powder, formulated milk liquid, and breast-milk substitute.

20. The method according to claim 13, wherein concentration of the NAD-related substance or a salt thereof acceptable for use in foods or pharmaceuticals at the time of ingestion is 1.9 μg/mL or higher in terms of nicotinamide mononucleotide (NMN) concentration.

21. The method according to claim 13, wherein concentration of the NAD-related substance or a salt thereof acceptable for use in foods or pharmaceuticals at the time of ingestion is 3.0 μg/mL or higher in terms of NMN concentration.

22. The method according to claim 13, wherein the composition is in the form of formulated milk powder and comprises 1.1 mg or more of the NAD-related substance or a salt thereof acceptable for use in foods or pharmaceuticals in terms of amount of NMN per 100 g of the composition.

23. The method according to claim 13, wherein the composition is in the form of formulated milk powder and comprises 1.8 mg or more of the NAD-related substance or a salt thereof acceptable for use in foods or pharmaceuticals in terms of amount of NMN per 100 g of the composition.

24. The method according to claim 13, wherein the NAD-related substance is NMN.
